(19) United States
(12) Patent Application Publication
(10) Pub. No.: US 2010/0114121 A1
(43) Pub. Date: May 6, 2010

(54) ANASTOMOSIS SUTURING DEVICE AND METHODS THEREOF

(75) Inventors: Dan Sade Hochstadter, Bet Alfa (IL); Nir Lilach, Kfar Yehoshua (IL); Eliahu Eliachar, Haifa (IL); Ofer Yossepowitch, Petach Tikvah (IL); Gideon Meyer-Brodnitz, Haifa (IL)

Correspondence Address: Fleit Gibbons Gutman Bongini & Bianco PL, 21355 EAST DIXIE HIGHWAY, SUITE 115, MIAMI, FL, 33180, US

(73) Assignee: KEREN MEDICAL LTD., Bet Alfa (IL)

(21) Appl. No.: 12/593,020

(22) PCT Filed: Mar. 19, 2008
(86) PCT No.: PCT/IL08/00389

§ 371 Date: Sep. 25, 2009

(30) Foreign Application Priority Data
Mar. 25, 2007 (IL) 182155

(51) Int. Cl. A61B 17/12

(52) U.S. Cl. 606/144

(57) ABSTRACT

The present invention provides an anastamosis suturing device (ASD), comprising an integrated suturing mechanism comprising a plurality of suturing wires, each of the wires is incorporated within a set of distal and proximal threading needles being positioned in a similar angular orientation, selected inter alia from 12′, 2′, 4′, 6′, 8′ and 10′ o'clock. the middle portion of each of the wires is arranged in a stack arrangement. Each needle is either operated separately or integrally with at least one another.

### FIELD OF THE INVENTION

The present invention generally relates to the urethra and bladder after removal of the prostate during a prostatectomy. Specifically, the invention relates to a method and a device for performing a urethral vesicle anastamosis. The invention is also intended for general use in tubular anastamosis, especially during open radical prostatectomy laparoscopic radical prostatectomy, and robotic assisted laparoscopic radical prostatectomy surgical procedures.

### BACKGROUND OF THE INVENTION

The prostate remains the second most common cause of cancer death. Because the incidence of prostate cancer increases more rapidly with age than does any other cancer and because the life expectancy is increasing, the number of men with prostate cancer and the number of deaths from the disease are expected to rise. Most prostate cancers detected nowadays are clinically localized and likely to be cured. A major treatment alternative for these patients includes radical prostatectomy (RP), which is the complete surgical removal of the prostate gland along with its facial coverings, requiring disconnection of the prostate from the urethra at the urogenital diaphragm and the urinary bladder at the bladder neck. To restore continuity of the urinary tract where the prostate has been removed, a new connection (anastomosis) of the bladder neck to the urethral stump must be accomplished. While the urethra-vesical anastamosis is generally technically challenging when surgeons use an open technique (open RP), it becomes remarkably difficult when applying the laparoscopic technique (laparoscopic radical prostatectomy LRP) with or without assistance of a robotic system (robotic assisted LRP) It is the attachment of the urethral stump to the bladder neck which is particularly difficult. This difficulty arises from several aspects, including the tendency of the urethral stump to retract proximally, as well as its delicate structure mandating placement of thin and accurate sutures to ascertain that sufficient urethral tissue is incorporated into the anastomosis without damaging the sphincteric mechanism. Further complicating this procedure is the fact that the urethral stump is located beneath the pubic bone thus requiring that the surgeon work at a difficult angle and in positions that are uncomfortable and limiting compared to open RP, laparoscopic surgeons face a two-dimensional screen further hindering their ability to place the urethral structures accurately.

A major impediment of radical prostatectomy remains its postoperative sequel related to the urethro-vesical anastomosis, which if not properly performed can lead to both urinary incontinence and outflow obstruction secondary to stricture at the anastomosis site.

Thus there remains a long felt need for the present invention relating to the reconnection of the urethra and bladder after a radical retropubic prostatectomy, specifically a method and device for performing an urethro-vesicle anastomosis.

### BRIEF DESCRIPTION OF THE FIGURES

In order to understand the invention and to see how it may be implemented in practice, a preferred embodiment will now be described, by way of non-limiting example only, with reference to the accompanying drawings;

FIG. 1 schematically illustrates the coaptation device fully assembled according to one embodiment of the present invention;

FIG. 2 schematically presenting an anastamosis suturing device (ASD), 1000 before it is inserted into the urethra according to yet another embodiment of the present invention;

FIG. 3 schematically presenting ASD, 1000 after it is inserted along the urethra 1001 and into the bladder according to yet another embodiment of the present invention;

FIG. 4A schematically illustrates ASD 1000 form the inner portion of the bladder, wherein penetration tip 10 perturbs inwardly according to yet another embodiment of the present invention; and,

FIG. 4B schematically illustrates needles after piercing of the bladder-neck and the urethra stump;

FIG. 5 schematically illustrates a plurality of the needle mechanism;

FIG. 6A-D schematically illustrate BnSM and UTM in the retracted actuators according to yet another embodiment of the present invention;

FIG. 7A-I schematically illustrate the different steps in the operation of ASD 1000 according to yet another embodiment of the present invention;

FIG. 8A-8B schematically illustrate the core of ASD 1000, comprising an integrated suturing mechanism (See FIG. 8A for example) comprising a plurality of suturing wires, each of said wires (See FIG. 8B for example) is incorporated within a set of distal and proximal threading needles being positioned in a similar angular orientation, selected inter alia from 12′, 2′, 4′, 6′, 8′ and 10′ o'clock; the middle portion of each of said wires is arranged in a stack arrangement; each needle is either operated separately or integrally with at least one another;

FIG. 9 schematically illustrates the connection between the suturing wire and the hollow suturing needle, according to a possible embodiment of the present invention; and,

FIG. 10 schematically illustrates in a non-limiting manner a perspective view of ASD 1000 according to yet another embodiment of the present invention; and

FIG. 11 schematically illustrates upper, front and side views of this embodiment.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications however, will remain apparent to those skilled in the art, since the generic principles of the present invention have been defined specifically to provide an apparatus for coaptation of the urethral stump to the neck of the bladder after removal of the prostate during a radical prostatectomy and method of performing urethral vesicle anastomosis thereby.

The term “coaptation” refers hereinafter to the joining of the bladder neck to the urethra after the prostrate has been removed in whole or in part.

The term “proximal” refers hereinafter to the extremity of the device closest to the glans of the patient's penis.

The term “distal” refers hereinafter to the extremity of the device furthest from the glans of the patient's penis.

The term “piercing” refers hereinafter in a non-limiting manner to a needle deployment motion through body tissue walls.

The term “bladderneck” or bladder-neck refers hereinafter to the proximal portion of the urethra remaining after the prostrate has been removed, and alternatively, to the opening of the bladder.

The term “plurality” applies hereinafter to any integer greater than one.

The term “needle” or “needles” apply hereinafter to a plurality of hollow needles, needles, harpoons or any other threading means

The invention generally pertains to an anastamosis suturing device (ASD, 1000), comprising an integrated suturing mechanism comprising a plurality of suturing wires 145, each of said wires is incorporated within a set of distal and proximal threading needles being positioned in a similar angular orientation, selected inter alia from 12′, 2′, 4′, 6′, 8′ and 10′ o'clock; the middle portion of each of said wires is arranged in a stack arrangement; each needle is either operated separately or integrally with at least one another.

The invention relates, according to one embodiment of eth invention, to ASD as defined above, characterized by a distal portion (100), located into the urethra and bladder, and a proximal portion (200), held by the surgeon; said distal portion comprises of an elongated rod-like member (rod, 1) with a main longitudinal axis; said rod comprising two concentric shafts, e.g., inner shaft 2 and outer shaft 3 and a plurality of operating modules, selected from a penetration tip 10 which is located in said rod's very distal end; at the distal portion of said rod, a plurality (e.g., two) suturing mechanisms is provided, namely a distal bladderneck suturing mechanism (BnSM, 20) and a proximal urethra threading mechanism (UTM, 30); said proximal portion (200) comprises of the operating mechanism, i.e., a BnSM's operating system (40), a UTM's operating mechanism (50) and a positioners operating mechanism (60); said BnSM's operating system (40) optionally comprises of an operating mechanism (e.g., one or more handles) 41, which is actuated by shaft 2 via coupling means 42; optionally, static gripping handle 43 facilitates operation of operating handle 41; said UTM's operating system (50) optionally comprises of an operating mechanism (e.g., one or more handles) 51, which is actuated by outer shaft 3 via coupling means 52; optional static gripping handle 53 facilitates operation of operating handle 51, those mechanical modules, and the said positioners operating mechanism, optionally rotate-able knob 62, are provided in a common chassis 44.

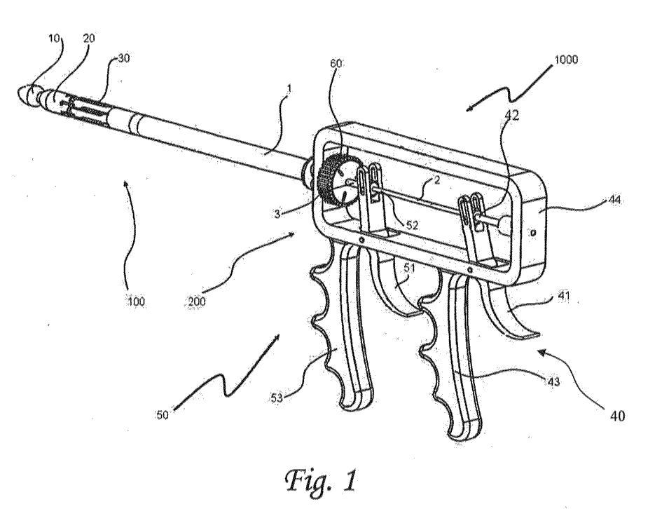

Reference is hence made to FIG. 1, presenting an anastamosis suturing device (ASD, 1000) according to one embodiment of the present invention. The ASD is characterized by a distal portion (100), located into the urethra and the bladder, and a proximal portion (200), held by the surgeon. The distal portion is having an elongated rod-like member (rod, 1) with a main longitudinal axis. The rod comprises two concentric shafts, i.e., inner shaft 2 and outer shaft 3. The rod further comprises or accommodates various operating modules, including inter alia a penetration tip 10, which is located in said rod's very distal end. Tip 10 can be made e.g., of elastic materials such as silicone, rubber or the like. At the distal portion of the rod, a series of two suturing mechanisms are provided, namely a distal bladderneck suturing mechanism (BnSM, 20) and a proximal urethra threading mechanism (UTM, 30). The proximal portion (200) comprises of the operating mechanism, i.e., a BnSM's operating system (40), a UTM's operating mechanism (50) and an positioners operating mechanism (60). The BnSM's operating system (40) comprises of an operating handle 41, which actuated inner shaft 2 by coupling means 42. It is in the scope of the invention, wherein a static gripping handle 43 facilitates easy operation of operating handle 41.

Similarly, The UTM's operating system (50) comprises of an operating handle 51, which actuated outer shaft 3 by coupling means 52. It is in the scope of the invention, wherein a static gripping handle 53 facilitates easy operation of operating handle 51. Those mechanical modules, and the aforesaid positioners operating mechanism, here rotetable knob 60, are provided in a common chassis 44.

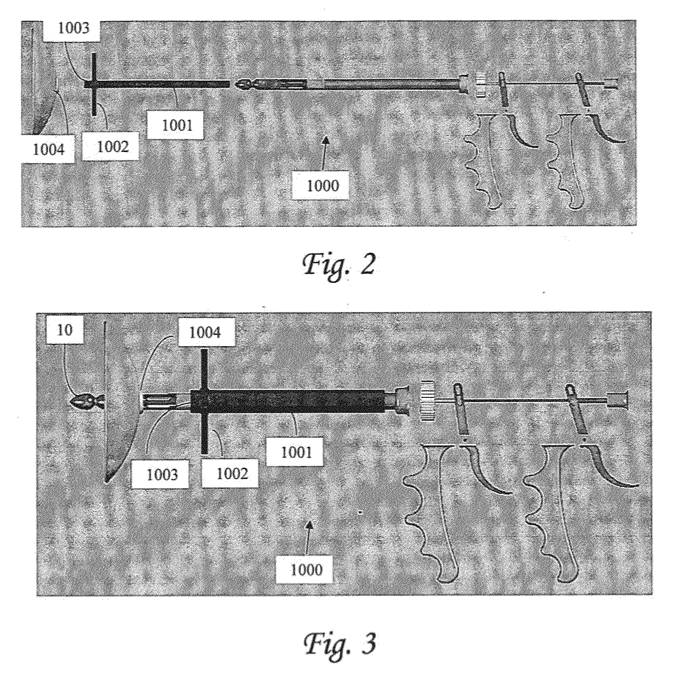

Reference is now made to FIG. 2, presenting ASD 1000 before it is inserted into the urethra. For convenience, the outer portion of the urethra, the abdomen wall, the inner portion of the urethra to be treated and the bladder neck shall be referred hereinafter 1001, 1002, 1003 and 1004, respectively.

Reference is now made to FIG. 3, presenting ASD, 1000 after it is inserted along the urethra 1001, via the bladderneck 100, such as the penetration tip 10 and BnSM, 20 (not shown) are located within the bladder.

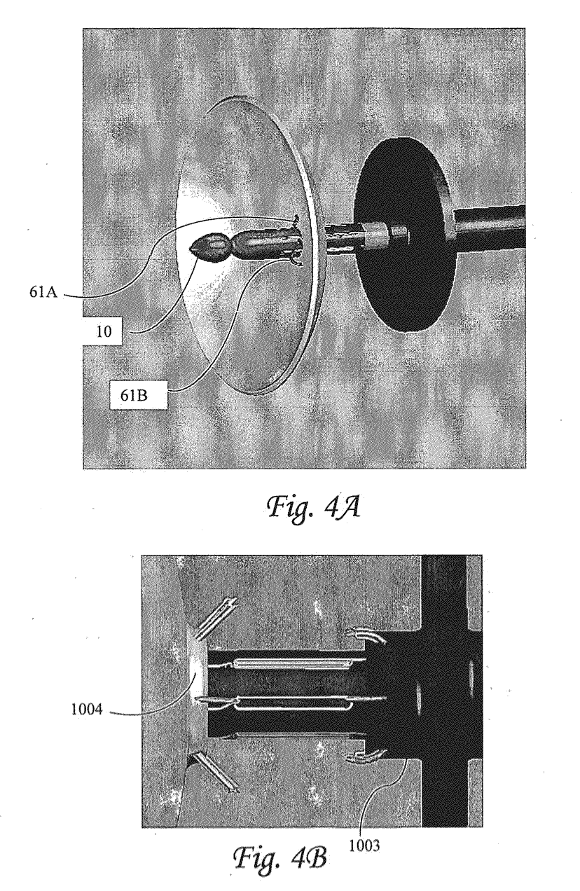

Reference is now made to FIG. 4A, ASD from the inner portion of the bladder, wherein penetration tip 10 perturbs inwardly. A plurality of positioners (61A, 61B etc) are maneuvered to their spread configuration by means of actuating the positioners operating mechanism (60, FIG. 1), e.g., by rotating the knob which connected to each of the positioners by a common positioners shaft (not shown here). Following the spreading of the positioners, the ASD is retracted retrogradely to a position where the positioners contact & firmly push the bladder wall. It is in the scope of the invention, wherein at least a portion of the positioners are one or more made of plastic, metal, Nitinol or inflatable balloon-like structures. Than, BnSM 40 (See FIG. 1) is activated.

After the bladderneck needles intruded, the UTM mechanism is activated. The UTM is located, possibly due to a mark on the shaft, in its position inside the urethra stump. Needles of the UTM are activated and pierce & thread the urethra stump.

Reference is now made to FIG. 4B, presenting both the activated UTM and BnSM, the needles extracted position.

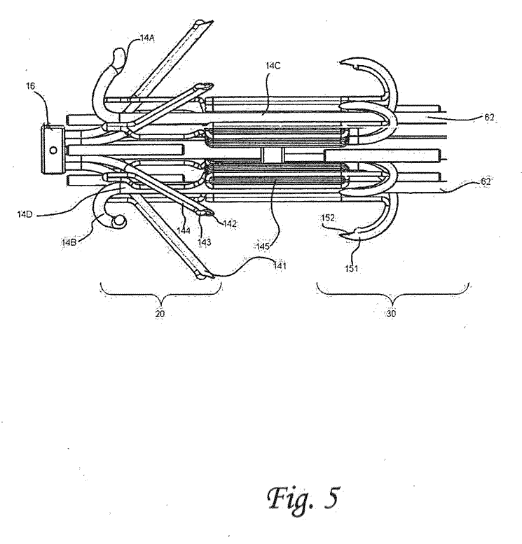

FIG. 5 is illustrating the integrated suturing mechanism of the present invention, with its BnSM 20 and UTM's 30 suturing mechanisms. Basically, the integrated suturing mechanism comprises of a first distal set of threading needles, i.e., the bladderneck needles, arranged in a predetermined positions, e.g., 12′, 2′, 4′, 6′, 8′ and 10′ o'clock, and a second proximal set of threading needle , i.e., the urethra needles, arranged in angular identical positions. Each of those pairs of distal and proximal needles of a mutual angular position is interconnected by a common thread or wire 145.

BnSM 20 is located within the bladder. It comprises of a plurality of N needles, N is any integer number equal or higher 1, e.g., 1, 2-6, 9 etc. The length of each of the needles is of a measure adapted to allow full piercing of the needle tip (141) through the bladderneck tissue. A thread or wire is reversibly connected to the tip portion of the needle, and the piercing is facilitated until the wire end protrudes to a significant measure from the pierced tissue. It is according to one embodiment of the invention, wherein needle's tip 141 comprises of a bore (142), e.g., being parallel to the needle's main longitudinal axis. The distal end of the wire is reversibly mounted within said bore (see 143). The wire is stacked in a suitable arrangement 145, such as a suture coil bay, and further connected to the UTM 30 suturing mechanism. The proximal end of wire is incorporated with needle 151, e.g., by mounted within a bore 152. The needles are preferably hollow needles.

The essence of this embodiment is hence a plurality of suturing wires, each of those wires is incorporated within a set of two threading needles, i.e., distal and proximal threading needles, being positioned in a similar angular orientation, e.g., 12′, 2′, 4′, 6′, 8′ and 10′ o'clock. The middle portion of each of those wires is arranged in a stack arrangement. It is in the scope of the invention wherein each needle is either operated separately or integrally with at least one additional. It is further in the scope of the invention where a first set of needles (e.g., three proximal needles in an orientation of 12′, 4′, 8′ o'clock) is simultaneously operated, and then, at least one second set is operated (e.g., three proximal needles in an orientation of 10′, 2′, 6′ o'clock) is simultaneously operated. Similarly may be the case in the distal needles.

It is in the scope of the invention wherein one or more of the sutures are individually coloured so that it is individually distinguished from the others in order to obtain the equivalent angular position of each suture on the urethral stump through the bladderneck wall. Possibly, each of the sutures additionally comprises of a smooth outer sheathing of a suitable texture for introduction into the urethral stump or the bladder neck. Alternatively, each of the sutures additionally comprises of a ferrule or suture lock on the free end of each suture to facilitate simple tying.

It is also in the scope of the invention, wherein the aforesaid anastamosis suturing device (ASD, 1000) assist the anastamosis during open surgery procedures; minimally invasive close surgery procedures; laparascopic procedures; robotic procedures or a combination thereof. More specifically, ASD 1000 is especially useful for coaptation of two separated tubular structures, namely proximal tubular structure and distal tubular structure in the mammalian body after removal of a section of the same. More specifically, ASD 1000 is useful for coaptation of urethra and bladder neck, for example in either radical or partial prostatectomy and/or anastamosis procedures.

The present invention also discloses a method for coaptation of two separated tubular structures, namely proximal tubular structure and distal tubular structure in the mammalian body after removal of a section of the same. More specifically, the invention discloses a method for coaptating of urethra and bladder neck, for example in either radical or partial prostectomy and/or anastamosis procedures.

The method comprising steps selected form a group consisting of (i) obtaining an ASD 1000; (ii) inserting the distal portion of the device via the urethra to the bladder of the patient, until the penetration tip is located within the bladder; (iii) spreading or inflating the positioners inside the bladder and pulling the device retrogradlly to ensure positioners effective attachment to the proximal portion of the inner bladder wall; (iv) piercing the bladderneck by a means of a set of N needles;(v) pulling/pushing the ASD towards the urethra to a predetermined location; e.g., up to a marked line (vi) piercing the urethra by a means of a set of N needles; (vii) retrieving both the bladder-neck's and urethra's needles back to the working tool; optionally, (viii) pulling the wires outwardly; (ix) folding the positioning mean; (x) extracting the ASD outside the body of the patient; (xi) by a means of a catheter inserted into the bladder through the urethra, pulling the bladder towards the urethra; and than (xii) tying or connecting each set of wires such that the bladder-neck and the urethra are well interconnected by means of those suturing wires. It is also in the scope of the invention wherein UTM's needles are activated before BnSM's needles.

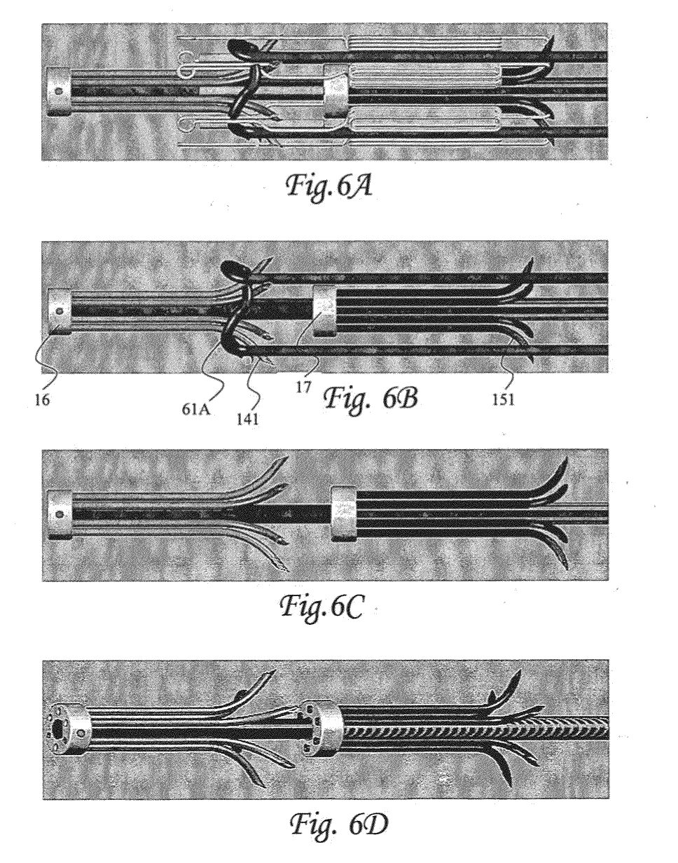

FIGS. 6A-6D shows the mechanism without the main suture BnSM and UTM actuators. FIG. 6A showing the mechanism complete, wherein FIG. 6B shows the same without the sutures. FIG. 6c shows the same without the sutures & the positioners. FIG. 6d shows the same without the sutures & the positioners from a different angle. BnSM's needles actuating (pushing) means (16) is adapted to push/pull one or more BnSM's needles (141) and UTM's needles actuating (pushing) means (17) is adapted to push/pull one or more UTM's needles (151). Positioners are presented as 61A. This actuation mechanism is provided here in a non-limiting manner whereas other actuation mechanism are possible, e.g., screw-based mechanisms; geared-actuators; motorized actuators; shape-based mechanism, such as Nitinol-based systems etc.

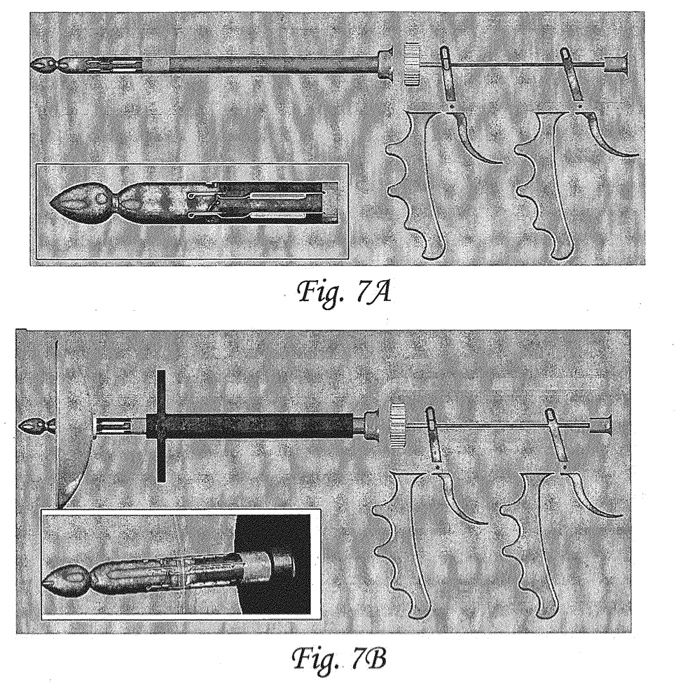

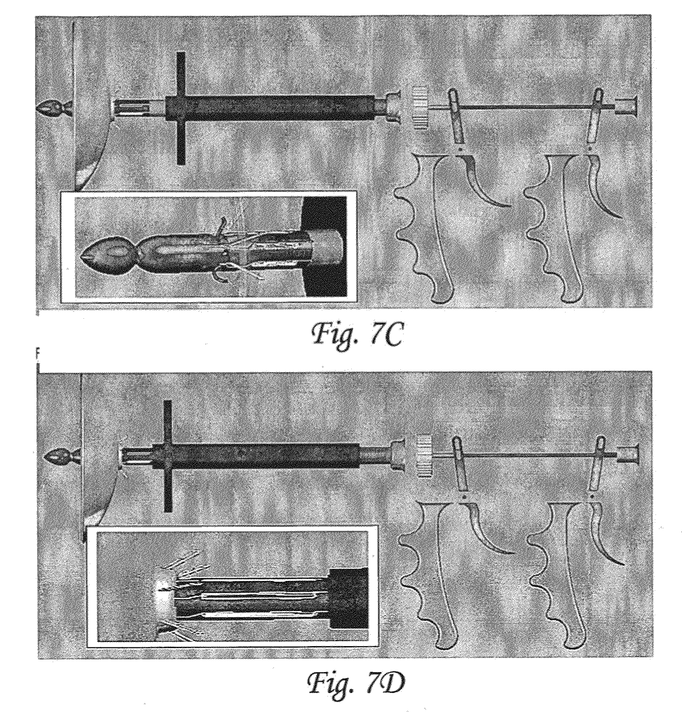

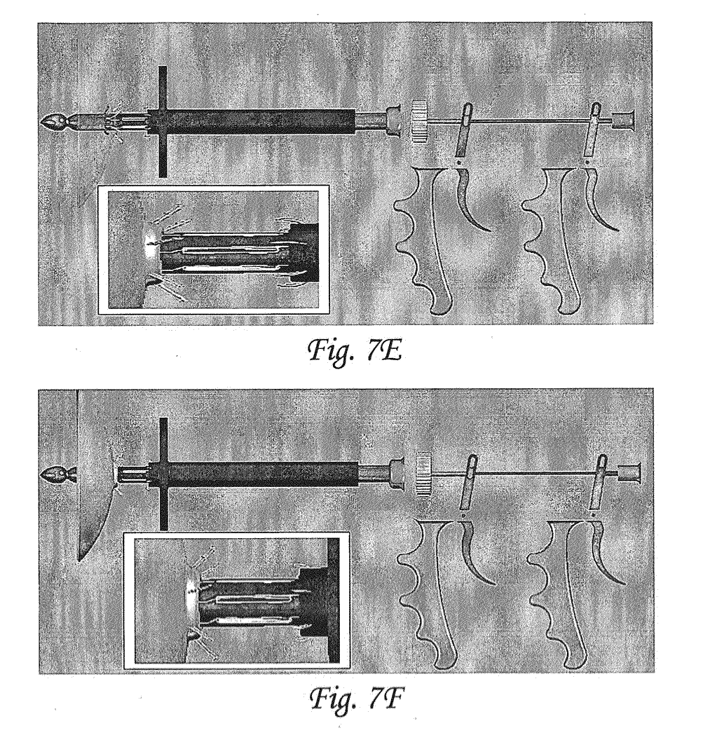

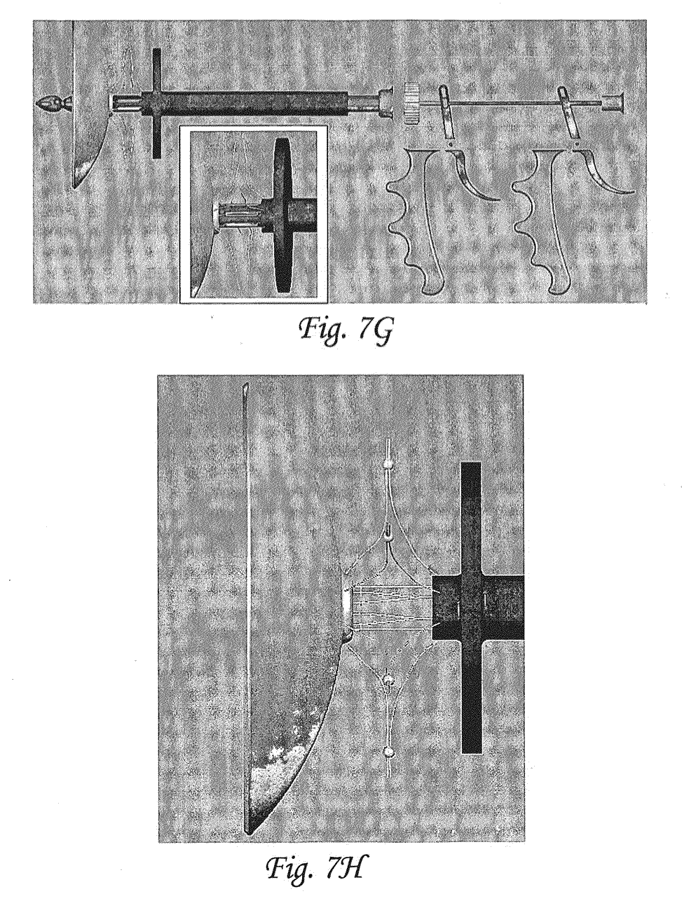

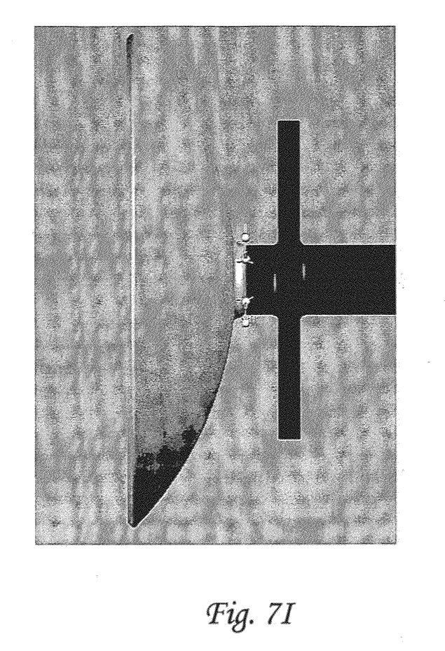

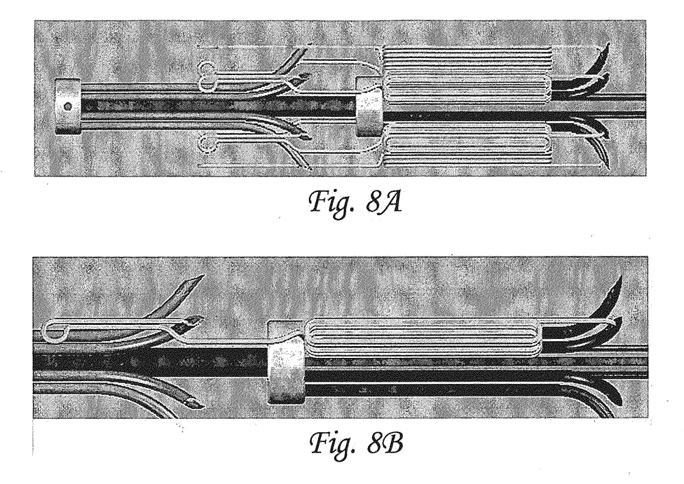

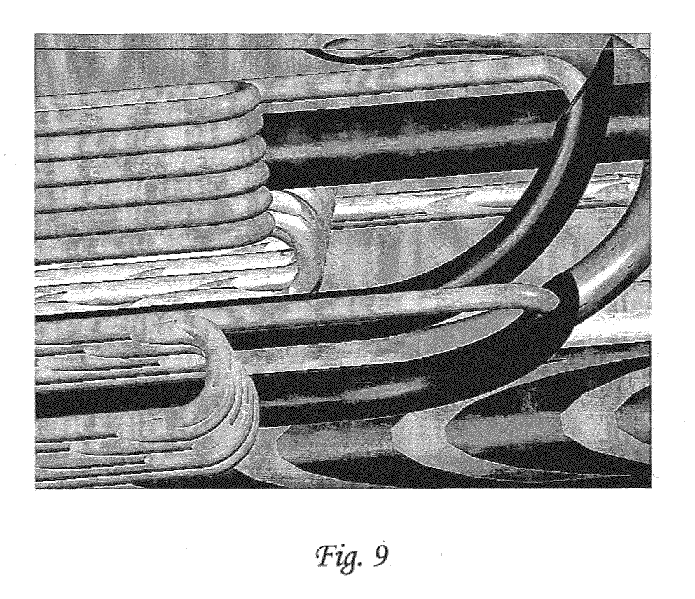

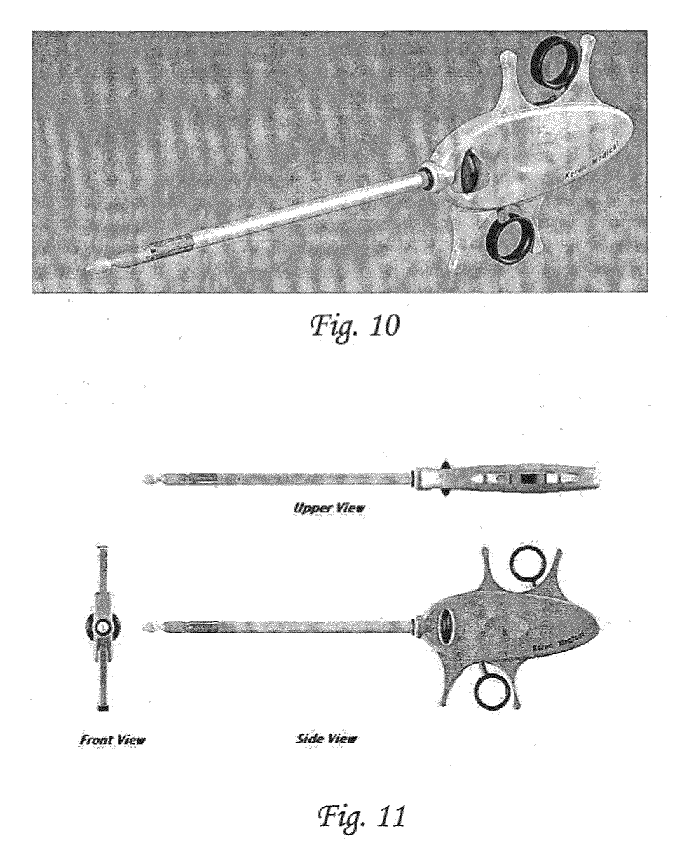

FIGS. 7A-7I illustrate the different steps in the operation of ASD 1000 according to one embodiment of the invention, wherein FIG. 7A illustrates the device before penetration; FIG. 7B illustrates the device after it penetrates the bladderneck; FIG. 7C front needle insertion; FIG. 7D illustrates urethra is pulled to back needle exit line; FIG. 7E illustrates both needles are out following piercing bladder-neck and the urethra stump; FIG. 7F illustrates needles are back in working tool and the sutures are left in place; FIG. 7G illustrates the sutures pulled outward; FIG. 7H illustrates bladder-neck & urethra with the suture ends coupled before tightening after removing the working tool; and FIG. 7I illustrates coaptation after tightening the sutures. It is well in the scope of the invention wherein the sutures are tied wherein ASD is removed form the body cavity. It is according to one embodiment wherein a catheter is inserted via the sutured urethra to the bladder, and sutures are then tied and tightened.

It is acknowledged in this respect that UTM may be activated before BnSM, BnSM before UTM and/or UTM and BnSM are activated simultaneously.

It is also in the scope of the invention, wherein the aforesaid method is adapted for anastamosis during open surgery procedures; minimally invasive close surgery procedures; laparascopic procedures; robotic procedures or a combination thereof.

1. An anastamosis suturing device (ASD, 1000), comprising an integrated suturing mechanism comprising a plurality of suturing wires 145, each of said wires is incorporated within a set of distal and proximal threading needles being positioned in a similar angular orientation, selected inter alia from 12′, 2′, 4′, 6′, 8′ and 10′ o'clock; the middle portion of each of said wires is arranged in a stack arrangement; each needle is either operated separately or integrally with at least one another.

2. The ASD according to claim 1, characterized by a distal portion (100), located into the urethra and bladder, and a proximal portion (200), held by the surgeon; said distal portion comprises of an elongated rod-like member (rod, 1) with a main longitudinal axis; said rod comprising two concentric shafts, e.g., inner shaft 2 and outer shaft 3 and a plurality of operating modules, selected from a penetration tip 10 which is located in said rod's very distal end; at the distal portion of said rod, a plurality (e.g., two) suturing mechanisms is provided, namely a distal bladderneck suturing mechanism (BnSM, 20) and a proximal urethra threading mechanism (UTM, 30); said proximal portion (200) comprises of the operating mechanism, i.e., a BnSM's operating system (40), a UTM's operating mechanism (50) and a positioners operating mechanism (60); said BnSM's operating system (40) optionally comprises of an operating mechanism (e.g., one or more handles) 41, which is actuated by shaft 2 via coupling means 42; optionally, static gripping handle 43 facilitates operation of operating handle 41; said UTM's operating system (50) optionally comprises of an operating mechanism (e.g., one or more handles) 51, which is actuated by outer shaft 3 via coupling means 52; optional static gripping handle 53 facilitates operation of operating handle 51, those mechanical modules, and the said positioners operating mechanism, optionally rotate-able knob 62, are provided in a common chassis 44.

3. Robotic ASD (1000) according to claim 2, wherein one or more of the BnSM and UTM are activated automatic or semi-automatic mechanisms.

4. The ASD (1000) according to claim 2, wherein said integrated threading mechanism comprising a first distal set of threading needles, i.e., BnSM's needles 20, arranged in any predetermined angular position, preferably selected from 12′, 2′, 4′, 6′, 8′ and 10′ o'clock, and a second proximal set of threading needlethreading needles, i.e., UTM's needles 30, arranged in identical angular positions, each of said angular pairs of distal and proximal needles of a mutual position is interconnected by a common thread or wire 145.

5. The ASD (1000) according to claim 2, wherein said BnSM, 20 is adapted to be temporarily inserted within the bladder, and comprises of a plurality of N needles, N is any integer number equal or higher than 1; the length of each of the needles is of a measure adapted to allow piercing of the needle tip (141) throughout the bladder neck tissue; a thread or wire 145 is reversibly connected to the tip portion of the needle, and the piercing is facilitated until the wire end protrudes to a significant measure from the pierced tissue; said wire is stacked in a suitable arrangement, and further connected to said UTM 30.

6. The ASD (1000) according to claim 2, wherein a first set of either BnSM or USM needles, e.g., in an orientation of e.g., 12′, 4′, 8′ o'clock is adapted to simultaneously operated, and then, at least one second set of either BnSM or USM needles are adapted to simultaneously operated, e.g., in an orientation of 10′, 2′, 6′ o'clock.

7. The ASD (1000) according to claim 2, wherein said BnSM's and/or said UTM's needles actuation mechanisms are selected from a group consisting of pull/push mechanism; screw-based mechanisms; geared-actuators; motorized actuators; shape-based mechanism, such as Nitinol-based systems or any combination thereof

8. A method of performing coaptation of urethra and bladder neck comprising steps selected form a group consisting of
i. obtaining an ASD 1000;
ii. inserting the distal portion of the device via the urethra into the bladder of a patient, until the penetration tip is located within the bladder;
iii. spreading or inflating the positioners inside the bladder and pulling the device retrogradlly to ensure positioners effective attachment to the proximal portion of the inner bladder wall;
iv. piercing the bladderneck by a means of a set of N needles and sutures;
v. pulling/pushing the ASD towards the urethra to a predetermined location; e.g., up to a marked line
vi. piercing the urethra by a means of a set of N needles;
vii. retrieving both the bladderneck's and urethra's needles back to the working tool;
optionally,
viii. pulling the wires outwardly;
ix. center folding the positioning mean;
x. pulling out the ASD outside the body of the patient;
xi. by a means of a balloon catheter inserted into the bladder throughout the urethra, to pull the bladder towards the urethra; and
xii. tying or otherwise connecting each set of wires such that the bladderneck and the urethra are well interconnected by means of those suturing wires.

9. The method according to claim 8, wherein the ASD is characterized by a distal portion (100), located into the urethra, and a proximal portion (200), held by the surgeon; said distal portion comprises of an elongated rod-like member (rod, 1) with a main longitudinal axis; said rod comprising two concentric shafts, i.e., inner shaft 2 and outer shaft 3 and a plurality of operating modules, selected inter alia from a penetration tip 10 which is located in said rod's very distal end; at the distal portion of said rod, a series of two suturing mechanisms is provided, namely a distal bladder neck suturing mechanism (BnSM, 20) and a proximal urethra threading mechanism (UTM, 30); said proximal portion (200) comprises of the operating mechanism, i.e., a BnSM's operating system (40), a UTM's operating mechanism (50) and an positioners operating mechanism (60); said BnSM's operating system (40) optionally comprises of an operating handle 41, which actuated inner shaft 2 by coupling means 42; static gripping handle 43 facilitates operation of operating handle 41; said UTM's operating system (50) optionally comprises of an operating handle 51, which actuated outer shaft 3 by coupling means 52; optional static gripping handle 53 facilitates operation of operating handle 51, those mechanical modules, and the said positioners operating mechanism, optionally rotetable knob 62, are provided in a common chassis 44; said ASD comprising an integrated suturing mechanism comprising a plurality of suturing wires 145, each of said wires is incorporated within a set of distal and proximal threading needles being positioned in a similar angular orientation, selected inter alia from 12′, 2′, 4′, 6′, 8′ and 10′ o'clock; the middle portion of each of said wires is arranged in a stack arrangement; each needle is either operated separately or integrally with at least one another.

10. The ASD according to claim 8, wherein at least a portion of said needles are pre-shaped;
especially made of superelastic (e.g. Nitinol made) alloys, adapted to retract and penetrate the tissue at a predetermined designed direction.

11. The ASD according to claim 8, wherein at least a portion of the needles of the BnSM is different by means of size or shape from at least a portion of the UTM.
